(19) United States
(12) Patent Application Publication
(10) Pub. No.: US 2026/0058019 A1
(43) Pub. Date: Feb. 26, 2026

(54) BACTERIAL ENGRAFTMENT DETERMINATION

(71) Applicant: Institute for Systems Biology, Seattle, WA (US)

(72) Inventors: Alex Carr, Seattle, WA (US); Christian Diener, Seattle, WA (US); Sean M. Gibbons, Seattle, WA (US)

(73) Assignee: Institute for Systems Biology, Seattle, WA (US)

(21) Appl. No.: 19/367,609

(22) Filed: Oct. 23, 2025

Related U.S. Application Data
(63) Continuation of application No. PCT/US2024/026898, filed on Apr. 29, 2024.
(60) Provisional application No. 63/462,768, filed on Apr. 28, 2023.

(51) Int. Cl. G16H 50/20

(52) U.S. Cl. CPC G16H 50/20

(57) ABSTRACT

Provided are computer-implemented methods, systems and products of determining bacterial engraftment in the gut of a subject.

### CROSS-REFERENCE TO RELATED APPLICATIONS

This application is a continuation of PCT Patent Application No. PCT/US2024/026898, filed on Apr. 29, 2024, which claims the benefit of and the priority to U.S. Provisional Patent Application No. 63/462,768, filed on Apr. 28, 2023. The entire disclosures of the aforementioned applications are incorporated by reference herein in their entireties for all purposes.

### ACKNOWLEDGEMENT OF GOVERNMENT SUPPORT

This invention was made with government support under R01 DK133468 awarded by the National Institute of Diabetes and Digestive and Kidney Diseases of the National Institutes of Health. The government has certain rights in the invention.

### FIELD

This disclosure relates to methods of determining bacterial engraftment of the gut and treatments.

### BACKGROUND

The human gut microbiome plays important roles in shaping host metabolism, in the development of chronic diseases, and in preventing opportunistic pathogen colonization and infection (refs 11-13). The metabolic versatility of gut bacteria allows for the stable coexistence of hundreds of commensal species within the gastrointestinal tract (ref 14). Some species extract energy and nutrition directly from indigestible dietary substrates, like plant fibers or recalcitrant proteins, while others subsist largely on host-derived mucosal glycans or on the vast array of metabolic byproducts produced by primary fiber, protein, and mucus degraders (refs 15, 16). Saturation of these metabolic niches by commensal microbes can prevent colonization and engraftment by external microbes that may share a similar niche, including pathobionts (refs 17, 18).

Perturbations to the gut microbiome (e.g., antibiotic use or diarrheal events) provide a window of opportunity for pathobiont colonization (ref 19), which could in turn lead to the development of disease following subsequent perturbations (refs 20, 21). One such pathobiont, Clostridioides difficile, is the most common hospital acquired gastrointestinal infection in the U.S. (refs 3, 4). C. difficile colonizes as much as 30-40% of community-dwelling adults without causing disease, lying in wait until the opportunity for infection arises (refs 1, 2). During active C. difficile infection (CDI), antibiotic treatment can be effective in suppressing C. difficile growth, but antibiotics also disrupt the ecology of the commensal microbiota and potentiate reinfection if C. difficile is not completely cleared by the treatment (refs 20, 21). Thus, an intact gut microbiota that prevents C. difficile colonization and engraftment is critical to the host's defense against CDIs (ref 13). This understanding has led to the widespread use of fecal microbiota transplants (FMTs) as a means of combating cases of recurrent CDI (rCDI), where antibiotic treatment proves insufficient (ref 7). While the biology of C. difficile has been fairly well-characterized in the context of disease, the pre-disease mechanisms of C. difficile colonization and engraftment are still poorly understood, as are the factors that govern C. difficile decolonization and FMT efficacy (ref 19).

There are currently no mechanistically grounded, generalizable approaches to accurately predicting the engraftment of an exogenous bacterial taxon in the context of a given microbiota. Previous work has leveraged machine learning (ML) to predict the engraftment of FMT donor strains in FMT recipients (ref 22). While effective and relatively accurate, this kind of quasi-black-box ML approach does not provide a means of understanding the molecular mechanisms that facilitate or prevent engraftment. Genome-scale metabolic models and classical flux balance analysis (FBA) have been invaluable tools for exploring how environmental conditions impact the metabolic capacity of individual bacterial taxa grown in vitro (ref 23). However, extending these methods to complex, multi-species communities has proved to be a challenge. Recently, an approach called cooperative tradeoff flux balance analysis (ctFBA) was reported to leverage microbiome compositional and dietary constraints to estimate steady-state community-scale metabolic fluxes (refs 24, 25).

### SUMMARY

Some embodiments of the present invention relate to computer-implemented methods for determining bacterial or pathobiont engraftment potential are provided. For example, a method according to an embodiment of the invention can include:
(a) accessing taxon abundance data of a gut microbiome of a subject;
(b) accessing a model configured to predict—using a flux balance analysis—dynamics of individual taxa of the gut microbiome of the subject and of engraftment bacteria or engraftment pathobiont, the model constrained by:
   (i) growth medium data representing extracellular substrate availability; and
   (ii) relative taxon abundance comprising the taxon abundance data of the gut microbiome of the subject in combination with taxon abundance of the engraftment bacteria or engraftment pathobiont set at a propagule pressure approximating an exposure or infection event;
(c) generating a prediction of an engraftment potential for the engraftment bacteria or engraftment pathobiont by processing the taxon abundance data of the gut microbiome of the subject using the model; and
(d) outputting the prediction of the engraftment potential of the engraftment or engraftment pathobiont bacteria for the subject.

The engraftment bacteria or engraftment pathobiont may be or may constitute the engraftment bacteria.

The model may be augmented with an intervention comprising one or more antimicrobials, prebiotics, probiotics, fecal microbiota transplants, dietary interventions, or a combination thereof.

The probiotic and the fecal microbiota transplant interventions may comprise treatment bacteria, and wherein the model is a microbial community-scale metabolic network model comprising a plurality of metabolic models for the individual taxa augmented with one or more metabolic models for the treatment bacteria having a taxon abundance approximating a gut exposure of interest.

Aan antimicrobial intervention may comprise one or more antibiotics, and wherein the taxon abundance of one or more susceptible taxa of the model are modified so as to approximate the antimicrobial activity of the one or more antibiotics.

The antibiotic may be selected from metronidazole, vancomycin, and fidaxomicin, and the antimicrobial activity is about half maximal effective concentration or greater.

The prebiotics and the dietary interventions may augment the growth medium data in an amount approximating a relative dosage of interest.

The prebiotic intervention may comprise, or may be selected from, soluble fiber such as inulin, pectin and psyllium, and insoluble fiber such as wheat bran, cellulose, lignin, and resistant starch, and the dietary intervention comprises, or is selected from, food intake, minerals, and vitamins.

The growth medium may be constrained by diet such as food type and food quantity, host metabolism such as by absorption of growth medium material in the small intestines, and one or more additional substrates selected from host molecules such as mucins and bile acids, vitamins, minerals, and prebiotics such as pectin and inulin.

A method disclosed herein may further comprise: generating an intervention efficacy score by comparing the predicted engraftment potential of the engraftment bacteria or engraftment pathobiont with and without the intervention. The intervention efficacy score may comprise a ratio of the predicted engraftment potential of the engraftment bacteria or engraftment pathobiont with and without the intervention.

The predicted engraftment potential may comprise: a growth rate; or a taxon abundance relative to a combination of the gut microbiome of the subject and the gut microbiome of the growth medium.

The propagule pressure approximating an exposure or infection event may be about 10% of the relative taxon abundance data.

A disclosed method can include displaying the predicted engraftment potential of the subject relative to an engraftment potential of a reference population.

The flux balance analysis can be a cooperative tradeoff flux balance analysis.

An objective function for the flux balance analysis can be configured to reward: community-wide growth corresponding to a full microbial community and taxon-specific growth specific to a given taxon.

An objective function for the flux balance analysis can be configured to reward: community-wide growth corresponding to a full microbial community and taxon-specific growth specific to a given taxon and production of short-chain fatty acids.

The engraftment bacteria or engraftment pathobiont can be one of: pathobiont bacteria, probiotic bacteria, fecal microbiota transplant (FMT) bacteria, or a combination thereof. The engraftment bacteria or engraftment pathobiont can comprise Clostridioides difficile or a mixture of strains thereof. Te Clostridioides difficile or a mixture of strains thereof can comprise, or can be selected from, a pan-genus model of Clostridioides representing common hypervirulent and non-epidemic strains, such as Clostridium difficile CD196, NAP07, NAP08, and R20291. The probiotic bacteria or engraftment pathobiont can comprise human gut commensal bacteria or a mixture of strains thereof. The human gut commensal bacteria or a mixture of strains thereof can be selected from Enterocloster bolteae, Anaerotruncus colihominis, Sellimonas intestinalis, Clostridium_Q symbiosum, Blautia sp001304935, Dorea_A longicatena, Clostridium_AQ innocuum, Flavonifractor plautii, Anaerobutyricum soehngenii, Akkermansia muciniphila, Anerobutyricum hallii, Clostridium beijernckii, Clostridium butyricum, Bifidobacterium infantis, and Generally Recognized as Safe (GRAS) bacterial strains. The fecal microbiota transplant (FMT) bacteria comprise, or can be selected from, OpenBiome FMTs.

Outputting the prediction may include outputting metabolite uptake and metabolite secretion of the engraftment bacteria or engraftment pathobiont relative to the gut microbiome of the subject and the growth medium.

The model may include a microbial community-scale metabolic network model (MCMM) generated by mapping the taxon abundance data of the subject to a plurality of metabolic models of the MCMM corresponding to the individual taxa of the subject.

Some embodiments of the present invention relate to computer-implemented methods for determining bacterial or pathobiont engraftment potential are provided. For example, a method according to an embodiment of the invention can include:
(a) accessing engraftment potential data for:
   (i) a gut microbiome of a subject simulated on a model configured to predict—using a flux balance analysis—dynamics of individual taxa of the gut microbiome of the subject and of engraftment bacteria or engraftment pathobiont, the model constrained by
      (1) relative taxon abundance comprising taxon abundance data of the gut microbiome of the subject in combination with taxon abundance of the engraftment bacteria or engraftment pathobiont set at a propagule pressure approximating an exposure or infection event,
      (2) growth medium data representing extracellular substrate availability from one or more different background diets, and
      (3) no intervention, or one or more interventions, the interventions comprising one or more antimicrobials, prebiotics, probiotics, fecal microbiota transplants, dietary interventions, or a combination thereof; and
   (ii) a plurality of gut microbiomes of a reference population comprising generally healthy individuals each individually simulated for engraftment potential on essentially the same one or more background diets as the subject, and optionally, essentially the same interventions as the subject;
(b) generating, for each of the one or more different background diets, a distribution based on engraftment potential of the subject and the reference population associated with the background diet and embedding the engraftment potential data of the subject associated with the background diet into a context of the distribution;
(c) generating, for each of the one or more different background diets, a comparative metric using the distribution for the background diet and the engraftment potential data of the subject simulated on the background diet; and
(d) identifying, based on the comparative metrics, a particular intervention to recommend for the subject, where the particular intervention includes a particular background diet, one or more particular interventions, or a combination thereof.

The comparative metric may be for a plurality of different background diets with and without the one or more interventions. A method disclosed herein may further generate a gut health report embedding the engraftment potential of the subject into a context of the distribution of the engraftment potential of the reference population for a given background diet, the gut health report identifying the particular intervention. Identifying the particular intervention may comprise ranking the interventions based on background diet. The background diets may comprise, or may selected from, a high-fiber diet such as a vegan high-fiber diet rich in resistant starch or a standard Mediterranean diet, a low fiber diet such as a standard European diet or a standard American diet, and a personalized diet.

It will be appreciated that, while some disclosures refer to engraftment bacteria or engraftment pathobiont, such embodiments may alternatively pertain to: only engraftment bacteria, only engraftment pathobiont, or a combination of engraftment bacteria and engraftment pathobiont. Also provided are computer-implemented systems and computer-program products for carrying out aspects of the disclosure.

In some embodiments, the system includes one or more data processors and a non-transitory computer readable storage medium containing instructions which, when executed on the one or more data processors, cause the one or more data processors to perform part or all of one or more methods disclosed herein.

In some embodiments, a computer-program product is provided that is tangibly embodied in a non-transitory machine-readable storage medium and that includes instructions configured to cause one or more data processors to perform part or all of one or more methods disclosed herein.

In some embodiments, a system is provided that includes one or more means to perform part or all of one or more methods or processes disclosed herein.

The present disclosure includes several advantages that correspond to (for example) providing practical applications and providing improved data processing (e.g., corresponding to more accurate predictions and/or more efficient data processing). For example, more accurate predictions can be provided of engraftment potential in a variety of different manners not captured by other approaches, including through MCMMs so as to provide detailed information on the ecological interactions within individual microbiota that prevent or facilitate engraftment, in addition to generating accurate, personalized engraftment predictions. Further advantages include the ability to facilitate practical applications by identifying potential interventions (such as probiotic treatments) for a given subject that is more likely to address a subject's condition as compared to current treatment-recommendation approaches. Further yet, embodiments of the invention can expand beyond merely identifying treatments to designing treatments. Additional advantages are apparent from the disclosure.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention as claimed has been specifically disclosed by embodiments and optional features, modification and variation of the concepts herein disclosed may be resorted to by those skilled in the art, and that such modifications and variations are considered to be within the scope of this invention as defined by the appended claims.

### BRIEF DESCRIPTION OF THE DRAWINGSS

The patent or application file contains at least one drawing executed in color. Copies of this patent or patent application publication with color drawing(s) will be provided by the Office upon request and payment of the necessary fee.

The present disclosure is described in conjunction with the appended figures:

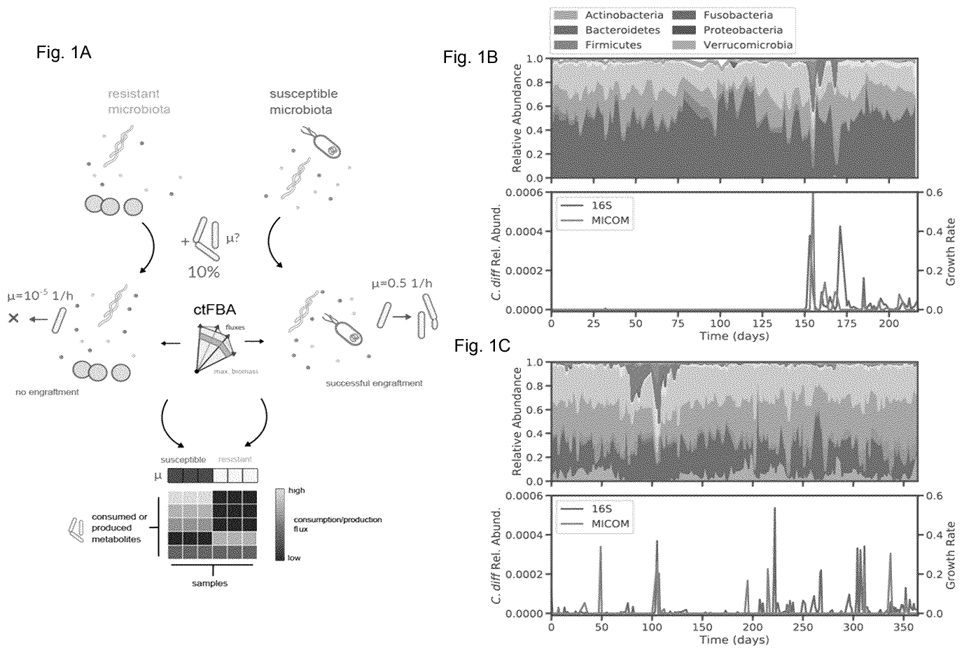

FIGS. 1A-1C show that in silico invasion assay accurately predicts C. difficile colonization in subject time series. (FIG. 1A) Schematic illustrating the in silico invasion assay workflow leveraged in this study. Personalized microbial community-scale metabolic models (MCMMs) are supplemented with 10% of a pan-genus Clostridioides model to simulate an invasion event and ctFBA was used to predict C. difficile engraftment and metabolic fluxes. (FIG. 1B) Donor a time series taken from David et al. displaying daily fluctuations in microbiome composition over a period of several months. Composition is displayed, colored by phylum-level annotations (different shading indicates taxonomic families). At day 150, Donor a experienced a diarrheal event and was subsequently colonized by C. difficile. Estimates of C. difficile relative abundance from 16S sequencing and predicted C. difficile growth rates (using MICOM) are displayed. (FIG. 1C) Time series from Donor B from the same study, who was apparently colonized by C. difficile (at very low relative abundances, near the limit of detection) throughout the sampling period.

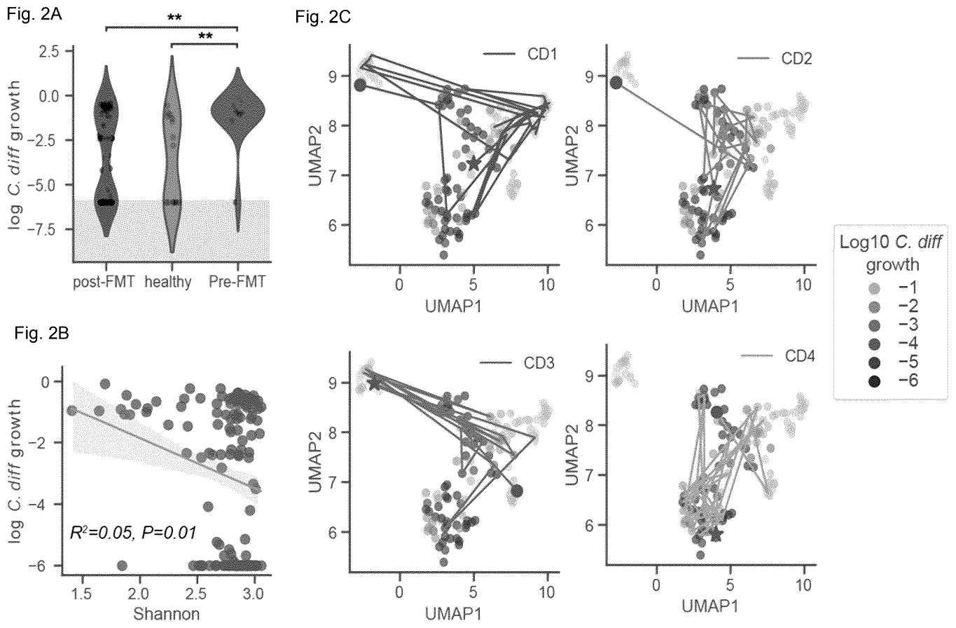

FIGS. 2A-2C show that the C. difficile growth rate predictions capture importance of community context for subject recovery from CDI. (FIG. 2A) Violin plots displaying predicted C. difficile log10 growth rate distributions across subject disease status (gray shading indicates the numerical accuracy of the simulation (values below 10-6 cannot be distinguished from zero and are considered negligible). Bars indicate comparisons for which differences were significant using the Welch test. *, P<0.05; **, P<0.01; ***, P<0.001. FIG. 2(B) Relationship between predicted C. difficile log10 growth rate and Shannon diversity. Ordinary least squares fit and 95% confidence interval are displayed, as well as regression R2 and p-value. (FIG. 2C) Two-dimensional representation of community import fluxes prior to in silico invasion using UMAP colored by log10 growth rates of C. difficile following in silico invasion. Subject trajectories are displayed, each with a red circle representing the subject's starting point (prior to FMT), and a red star representing the subject's end point (post recovery).

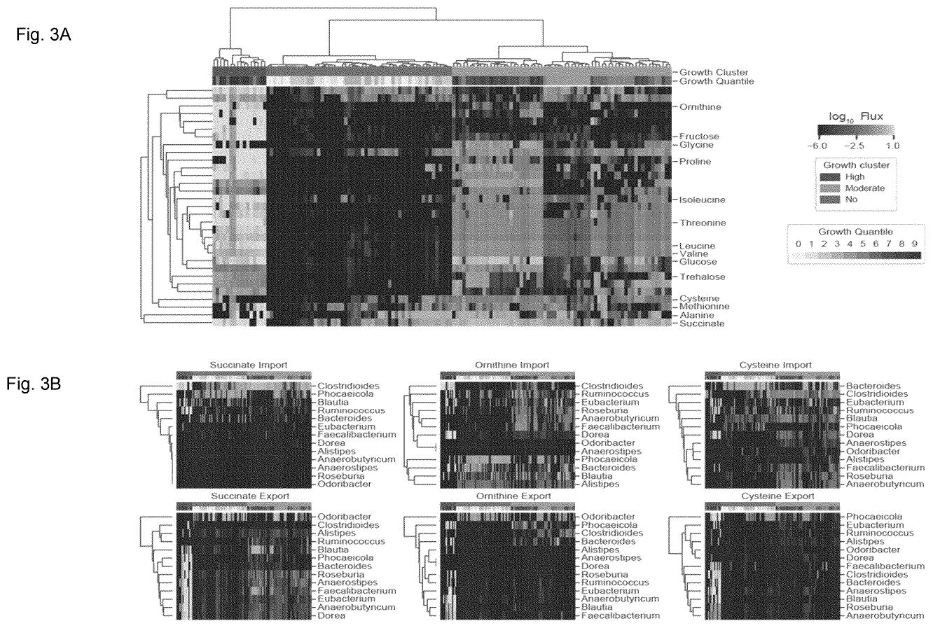

FIGS. 3A-3B show that C. difficile occupies multiple metabolic niches across communities. (FIG. 3A) Biclustered C. difficile log10 import fluxes, where each row is the import flux of a particular metabolite and each column is a subject sample. Imports for which the log variance across samples was >=4.5 are displayed as the blue-to-yellow heatmap. (FIG. 3B) Biclustered community import and export fluxes of specific metabolites associated with C. difficile colonization, where each row is a genus and each column is a subject sample. Genera for which the mean import or export flux across samples was >10-6 are displayed. Fluxes across samples are displayed using blue-to-yellow heatmap coloring. C. difficile log10 growth rate quantiles are displayed in white-to-red heatmap coloring for each subject sample in the top row of each plot. Additionally, three coarse grain growth clusters are noted. These growth clusters represent “high growth”, “moderate growth”, and “no growth” phenotypes.

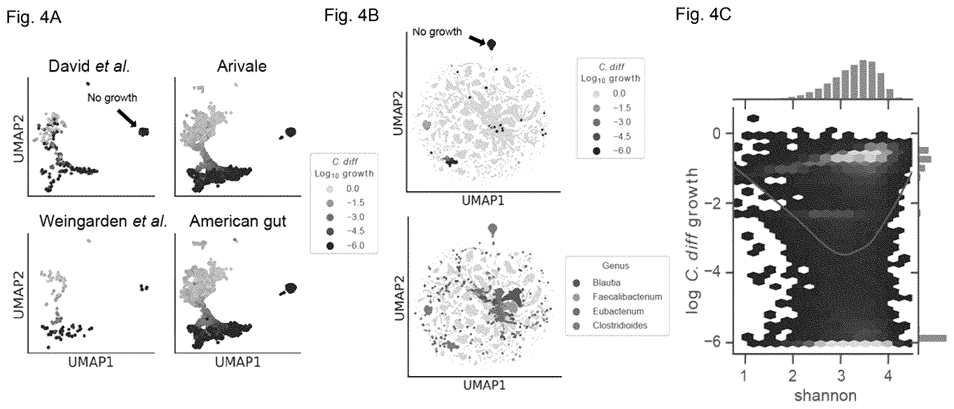

FIGS. 4A-4C show Growth niches in large healthy cohorts challenged with C. difficile. (IFG. 4A) Two dimensional representation of log10 C. difficile import fluxes using UMAP across four independent data sets. Colors denote C. difficile growth rate ranging from low (blue) to high (yellow). The position of the no growth cluster is indicated. (FIG. 4B) Two dimensional representation of log10 genus import fluxes using UMAP across all datasets. Top panel displays log10 C. difficile growth rate within the context of all other genera. Bottom panel colors C. difficile and three genera of interest: Blautia, Faecalibacterium, and Eubacterium. The position of the no growth cluster is indicated. (FIG. 4C) Two-dimensional hexagonal binning of log10 C. difficile growth rate and community alpha diversity (Shannon index). Red trend line indicates a LOWESS fit to the log10 C. difficile growth rate and community Shannon diversity data.

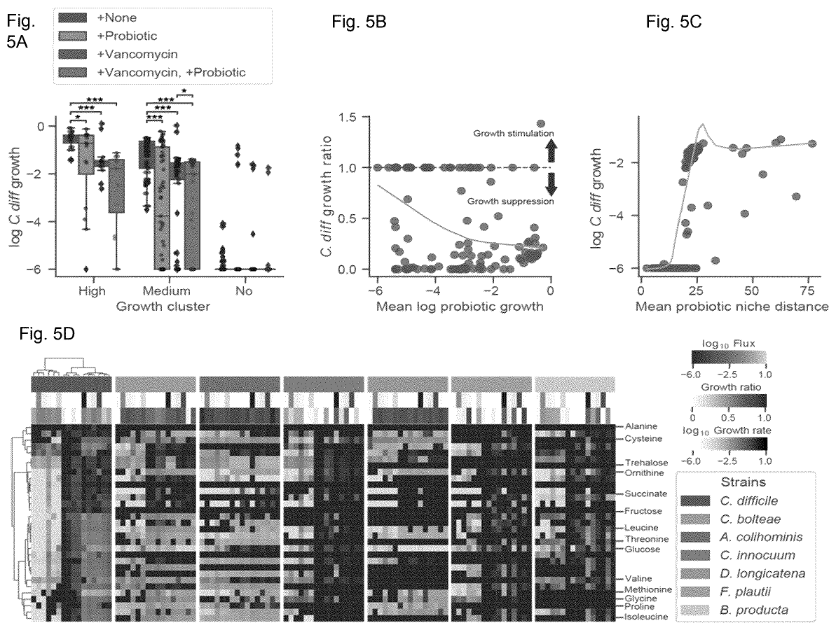

FIGS. 5A-5D show that simulated probiotic intervention effectively suppresses C. difficile growth in silico. (FIG. 5A) Box plots displaying log10 C. difficile growth rate across growth clusters and simulated interventions. Growth clusters are those identified by bi-clustering of C. difficile import fluxes using the Weingarden data (FIG. 3). Conditions include +None (no intervention control), +Probiotic (introduction of 6 strain probiotic previously identified as an effective treatment for CDI at a total relative abundance of 50% equally distributed across the strains), +Vancomycin (90% reduction of C. difficile relative abundance as well as all genera known to be impacted by Vancomycin), and +Vancomycin, +Probiotic (introduction of 6 strain probiotic in combination with simulated vancomycin treatment). Bars indicate comparisons for which differences were significant using the Wilcoxon signed-rank test. *, P<0.05; **, P<0.01; ***, P<0.001. (FIG. 5B) Relationship between C. difficile growth ratio and mean log10 probiotic growth rate. C. difficile growth ratio is the growth rate of samples in the +Vancomycin, +Probiotic intervention relative to +None. Values below 1 indicate growth suppression by the probiotic and values above 1 indicate growth stimulation. The dashed line marks the value at which no effect is observed (1). Orange trend line indicates a LOWESS fit to the C. difficile growth ratio and mean log10 probiotic growth rate. (FIG. 5C) Relationship between log10 C. difficile growth rate and mean probiotic niche distance. Niche distance was calculated using the Euclidean distance of log10 import flux vectors of each probiotic strain relative to C. difficile on a per sample basis. Orange trend line indicates a LOWESS fit to the log10 C. difficile growth rate and mean probiotic niche distance. (FIG. 5D) Biclustered log10 import fluxes for C. difficile and probiotic strains for samples previously identified as “high growth”, where each row is the import flux of a particular metabolite and each column is a subject sample. Imports displayed are those previously identified as important for C. difficile. Color bars indicate sample C. difficile growth ratio and strain specific log10 growth rate. Ordering of samples and metabolites is the same across heatmaps and based on biclustering of C. difficile data.

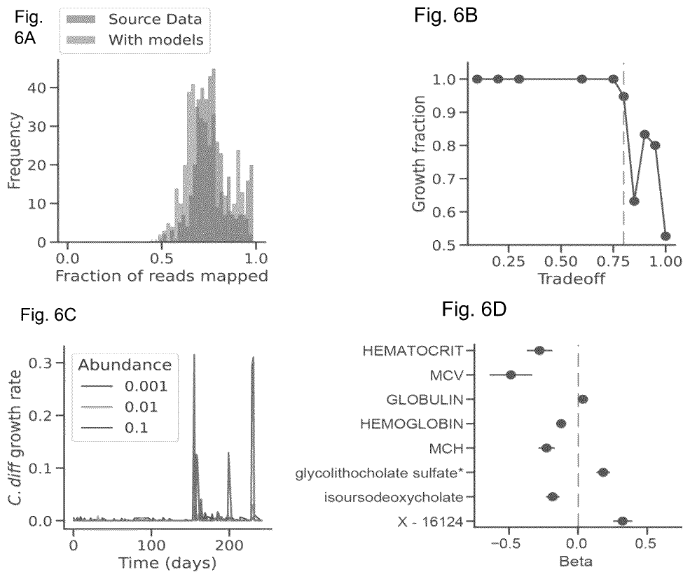

FIGS. 6A-6D show development of in silico C. difficile invasion assay. (FIG. 6A) Histograms displaying the fraction of reads mapped at the genus level for the David et al. 16S amplicon data using an NCBI reference and genus level metabolic models using the AGORA database referred to as “Source Data” and “With models” respectively. (FIG. 6B) Median growth fraction across samples (e.g., fraction of taxa with estimated growth rate>10-6) as a function of model tradeoff value. Dashed line indicated the tradeoff value chosen for subsequent analyses. (FIG. 6C) Relationship between C. difficile invasion abundance and growth rate for one of the two David et al. time series. (FIG. 6D) Association coefficients for estimated C. difficile log growth rate, blood metabolite concentrations, and clinical labs for the Arivale cohort.

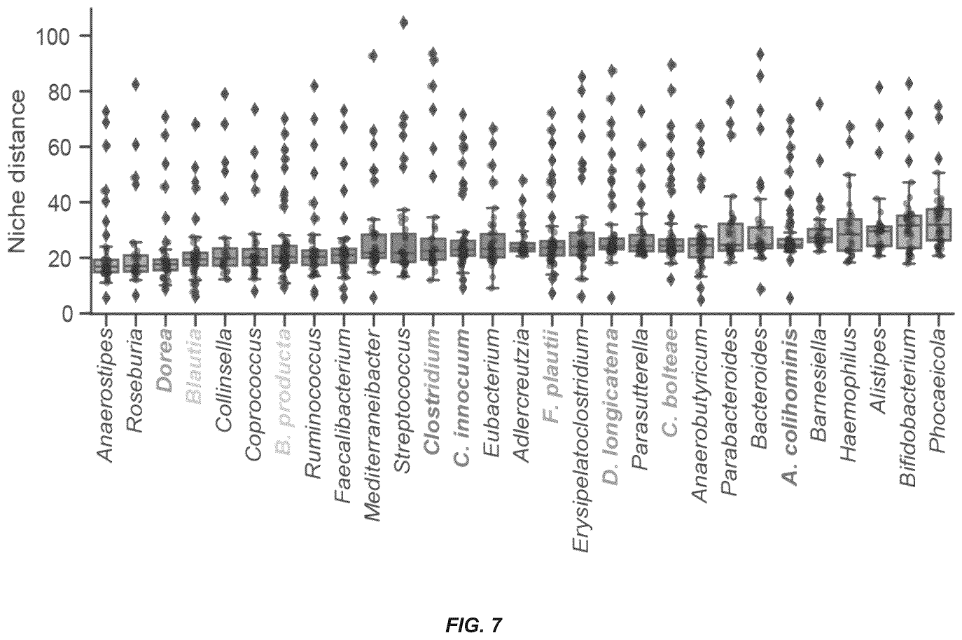

FIG. 7 shows probiotic strains and associated genera have niche distances close to C. difficile relative to unrelated genera. Niche distances of strains and genera, represented as the Euclidean distance between flux vectors, relative to C. difficile across CDI-FMT cohort samples for which the C. difficile growth rate>10-6. Genera and strains are ordered by the median niche distance. Probiotic strains and associated genera are colored, consistent with the legend in FIG. 5D. C. bolteae and C. innocuum are both members of the genus Clostridium.

### DESCRIPTION OF SPECIFIC EMBODIMENTS

As summarized above, provided are computer-implemented methods, systems, computer-program products and other products for determining bacterial engraftment in the gut of a given subject as well as interventions.

The disclosure is exemplified by leveraging community-scale metabolic modeling to facilitate personalized Clostridioides difficile engraftment predictions and precision probiotic efficacy assessment in the human gut. For example, amplicon data (e.g., e 16S amplicon data) can be leveraged with C. difficile colonization dynamics and/or ametabolic modeling framework (e.g., configured to infer metabolic interactions in gut microbiota), such as MICOM (ref 24), to build and test a model specifically designed to estimate bacteria engraftment potential (e.g., C. difficile engraftment potential) within a given microbiome and dietary context. The model may be configured to use flux balance analysis to predict dynamics pertaining to an engraftment process and/or a variable predicting an occurrence or extent of engraftment at a particular time point.

In various embodiments of the invention, novel insights into how C. difficile can occupy three discrete metabolic niches across individuals (e.g., as well as what metabolic interactions within gut communities promote or prevent colonization) are used to generate novel computer-implemented methods, systems, uses, means, computer-readable media, etc. In some embodiments, it may predicted and/or determined that a given individual is infected with C. difficile, which may trigger (or conditionally trigger) an absolute or probabilistic classification of the individual as being a responder or non-responders to probiotic cocktails for the treatment of rCDI (ref 9).

Computer-implemented methods, systems, computer program products and other products of the disclosure provide a novel approach towards predicting C. difficile engraftment risk as well as treatments that are predicted to be effective in treating individual subjects. This includes application in the design of precision dietary or probiotic interventions aimed at decolonizing individuals who are already carrying C. difficile and preventing engraftment in those who are not current carriers. Embodiments of the subject invention may further include or relate to discovering other pathobionts beyond C. difficile, probiotic bacterial strains, or for entire microbial consortia (e.g., FMTs from different donors), and the same or different embodiments may use such pathobionts, strains, or consortia to facilitate treatment, health improvement/stabilization, etc. As one example, one or more particular diet modifications and/or one or more supplements may be identified and availed based on a discovered pathobionts, strains, or consortia.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

In further describing the subject invention, certain terms used in accordance with the invention are described first in greater detail, followed by a description of methods, systems and products, followed by examples of the disclosure.

### I. Terms

Definitions of common terms in computational and data science may be found in: Ranganathan et al. (2018) Encyclopedia of Bioinformatics and Computational Biology: ABC of Bioinformatics, Elsevier (which is hereby incorporated by reference in its entirety for all purposes); Saltz et al. (2017) An introduction to data science, Sage Publications (which is hereby incorporated by reference in its entirety for all purposes); James et al. (2013) An introduction to statistical learning, (Vol. 112, p. 18) New York, Springer (which is hereby incorporated by reference in its entirety for all purposes); and other similar references.

### II. Computer-Implemented Methods, Systems and Products

As summarized above, provided are exemplary computer-implemented methods, systems and products for determining bacterial engraftment in a subject and predicting an impact of one or more potential interventions.

Some embodiments include: a computer-implemented method of determining bacterial engraftment of a subject, the computer comprising one or more processors programmed to perform a series of steps, comprising:
(a) accessing taxon abundance data of a gut microbiome of a subject;
(b) accessing a model configured to predict—using a flux balance analysis—dynamics of individual taxa of the gut microbiome of the subject and of engraftment bacteria or engraftment pathobiont, the model constrained by:
   (i) growth medium data representing extracellular substrate availability; and
   (ii) relative taxon abundance comprising the taxon abundance data of the gut microbiome of the subject in combination with taxon abundance of the engraftment bacteria or engraftment pathobiont set at a propagule pressure approximating an exposure or infection event;
(c) generating a prediction of an engraftment potential for the engraftment bacteria or engraftment pathobiont by processing the taxon abundance data of the gut microbiome of the subject using the model; and
(d) outputting the prediction of the engraftment potential of the engraftment or engraftment pathobiont bacteria for the subject.

Some embodiments include: a computer-implemented method comprising:
(a) accessing engraftment potential data for:
   (i) a gut microbiome of a subject simulated on a model configured to predict—using a flux balance analysis—dynamics of individual taxa of the gut microbiome of the subject and of engraftment bacteria or engraftment pathobiont, the model constrained by
      (1) relative taxon abundance comprising taxon abundance data of the gut microbiome of the subject in combination with taxon abundance of the engraftment bacteria or engraftment pathobiont set at a propagule pressure approximating an exposure or infection event,
      (2) growth medium data representing extracellular substrate availability from one or more different background diets, and
      (3) no intervention, or one or more interventions, the interventions comprising one or more antimicrobials, prebiotics, probiotics, fecal microbiota transplants, dietary interventions, or a combination thereof; and
   (ii) a plurality of gut microbiomes of a reference population comprising generally healthy individuals each individually simulated for engraftment potential on essentially the same one or more background diets as the subject, and optionally, essentially the same interventions as the subject;
(b) generating, for each of the one or more different background diets, a distribution based on engraftment potential of the subject and the reference population associated with the background diet and embedding the engraftment potential data of the subject associated with the background diet into a context of the distribution;
(c) generating, for each of the one or more different background diets, a comparative metric using the distribution for the background diet and the engraftment potential data of the subject simulated on the background diet; and
(d) identifying, based on the comparative metrics, a particular intervention to recommend for the subject, where the particular intervention includes a particular background diet, one or more particular interventions, or a combination thereof.

In one embodiment, the model may predict a presence or extent of engraftment. The model may include (for example) a microbial community scale metabolic network model (MCMM) and/or microbial community scale metabolic network model (MICOM). The model may be configured to simulate metabolic interactions within a community of different microbial species. The model may be configured to predict how microbes in a metabolic network interact, compete and/or cooperate in an environment. The model may be configured to predict taxom-specific and/or community-wide variables in view of environmental variables. Environmental variables may include (for example): initial or stable nutrient availability; one or more physical conditions (e.g., temperature, pH, oxygen levels, and/or salinity); kinetic parameters (e.g., rates of metabolic reactions); etc.

The model may use and/or include flux balance analysis, such cooperative tradeoff flux balance analysis. Flux balance analysis is configured to optimize a particular object (e.g., biological or biochemical objective). Cooperative tradeoff flux balance analysis may prioritize and/or reward multiple variables, such as community-wide/individual growth (corresponding to a full microbial community), taxon-specific growth (specific to a given taxon) and/or production of short-chain fatty acids. It will be appreciated that community-wide/individual growth can represent a degree to which a community of bacteria (i.e., a group of different bacterial speciese that live and interact within a shared environment) grows (e.g., across simulated time points).

The model may have been trained using data corresponding to subjects that have experienced and/or been diagnosed with C. difficile infection and/or recurrent C. difficile infection. Additionally or alternatively, the model may be configured to generate predictions that assume that a subject has experienced at least a threshold number of C. difficile infections (e.g., at least one, at least two, at least five infections) or that has or has been diagnosed with recurrent C. difficile infection.

It will be appreciated that disclosures presented herein that relate to C. difficile may be expanded or altered to additionally or alternatively relate to other pathobionts. For example, in some embodiments, a model disclosed herein (e.g., that uses flux balance analysis, cooperative tradeoff flux balance analysis, etc.) may be configured to predict production of, existence of, or amount of one or more other microbial metabolites (e.g., short-chain-fatty-acids, hydrogen sulfide or trimethylamine N-oxide). Thus, disclosed embodiments present a new path forward in engineering the ecological composition and metabolic outputs of microbiota to prevent and treat disease.

Implementation of, execution of, and/or assessment of the model may be augmented with recommending and/or initiating an intervention comprising (for example) one or more antimicrobials, prebiotics, probiotics, fecal microbiota transplants, dietary interventions, or a combination thereof. In certain embodiments, the intervention further comprises generating an intervention efficacy score by comparing the engraftment potential of the engraftment bacteria with and without the intervention. a specific example is where the intervention efficacy score comprises a ratio of the engraftment potential of the engraftment bacteria with and without the intervention.

An intervention of specific interest is where the probiotic and the fecal microbiota transplant interventions comprise treatment bacteria, and wherein the model is augmented with one or more genome-scale metabolic models (GEMs)for the treatment bacteria having a taxon abundance approximating a gut exposure of interest.

Another intervention of specific interest is where the antimicrobial intervention comprises one or more antibiotics, and wherein the taxonomic abundance of one or more susceptible taxa represented in the metabolic model are modified so as to approximate the antimicrobial activity of the one or more antibiotics. In some embodiments, the antibiotic is selected from metronidazole, vancomycin, and fidaxomicin, and the antimicrobial activity is about half maximal effective concentration or greater.

An additional example of the intervention is where the prebiotics and the dietary interventions augment the growth medium data in an amount approximating a relative dosage of interest. In other embodiments, the prebiotic intervention comprises, or is selected from, soluble fiber such as inulin, pectin and psyllium, and insoluble fiber such as wheat bran, cellulose, lignin, and resistant starch, and the dietary intervention comprises, or is selected from, food intake, minerals, and vitamins.

Generally, the growth medium can be constrained by diet such as food type and food quantity, and host metabolism such as by absorption of growth medium material in the small intestines. In many embodiments, the growth medium is further constrained by one or more additional substrates selected from host molecules such as mucins and bile acids, vitamins, minerals, and prebiotics such as pectin and inulin.

As can be appreciated, flux balance analysis is a mathematical method for simulating metabolism in genome-scale reconstructions of metabolic networks. A preferred flux balance analysis of the disclosure is cooperative tradeoff flux balance analysis. The cooperative tradeoff flux balance analysis typically comprises a cooperative tradeoff parameter set to allow about 60-90% of all GEMs to grow in the absence of the engraftment bacteria.

In certain embodiments, the engraftment potential comprises, or is selected from, growth rate and taxon abundance relative to the gut microbiome of the subject and the growth medium.

A featured aspect of the disclosure is applying in the model (e.g., MCMM or model that uses flux-balance analysis) a propagule pressure for the engraftment bacteria approximating an exposure or infection event. As referred to herein, “propagule pressure” is intended a composite measure of the number of individuals of a species released into a region of interest, and more specifically, a composite measure of the number of individuals of a species released into a region to which they are not native. For example, the propagule pressure approximating an exposure or infection event in a model of the disclosure is about 1-20%, generally about 5-15%, usually typically about 8-12%, and typically about 10% of the relative taxon abundance data for an engraftment bacteria such as C. difficile. As such, in certain embodiments, the propagule pressure approximating an exposure or infection event is about 10% of the relative taxon abundance data. Typically, propagule pressure incorporates estimates of the absolute number of individuals involved in any one release event and the number of discrete release events.

In certain embodiments, the method of determining bacterial engraftment further comprises displaying the engraftment potential of the subject relative to the engraftment potential of a reference population.

In certain embodiments, the engraftment bacteria is selected from pathobiont bacteria, probiotic bacteria, fecal microbiota transplant bacteria, or a combination thereof. An example of pathobiont bacteria is Clostridioides difficile or a mixture of strains thereof. a specific example is where the Clostridioides difficile or a mixture of strains thereof comprise, or are selected from, a pan-genus model of Clostridioides representing common hypervirulent and non-epidemic strains, such as Clostridium difficile CD196, NAP07, NAP08, and R20291. Probiotic bacteria of particular interest comprise human gut commensal bacteria or a mixture of strains thereof. Specific examples are where the human gut commensal bacteria or a mixture of strains thereof are selected from Enterocloster bolteae, Anaerotruncus colihominis, Sellimonas intestinalis, Clostridium_Q symbiosum, Blautia sp001304935, Dorea_A longicatena, Clostridium_AQ innocuum, Flavonifractor plautii, Anaerobutyricum soehngenii, Akkermansia muciniphila, Anerobutyricum hallii, Clostridium beijernckii, Clostridium butyricum, Bifidobacterium infantis, and Generally Recognized as Safe (GRAS) bacterial strains. In some embodiments, the fecal microbiota transplant (FMT) bacteria comprise, or are selected from, OpenBiome FMTs.

Also included are embodiments where the method of determining bacterial engraftment further comprises outputting metabolite uptake and metabolite secretion of the engraftment bacteria relative to the gut microbiome of the subject and the growth medium.

The disclosure also includes a system as well as a computer-program product. In general, the system comprises (i) one or more data processors, and (ii) a non-transitory computer readable storage medium containing instructions which, when executed on the one or more data processors, cause the one or more data processors to perform a set of actions to perform part or all of one or more methods disclosed herein. Also in general, the computer-program product is tangibly embodied in a non-transitory machine-readable storage medium, including instructions configured to cause one or more data processors to perform a set of actions to perform part or all of one or more methods disclosed herein.

### EXAMPLES

The following examples are provided to illustrate certain particular features and/or embodiments. These examples should not be construed to limit the disclosure to the particular features or embodiments described.

### Example 1: External Data Collection and Processing

Data used in this study came from four sources. This included both cross sectional and time series 16S amplicon sequence data from David et al., Weingarden, A. et al., the American Gut (McDonald, D. et al.), and a former scientific wellness program run by Arivale, Inc. (refs 27,29,31,42). Publicly available 16S amplicon sequence data and associated metadata were downloaded from the sequence read archive (SRA). Additionally, de-identified 16S amplicon sequence data, associated metadata, and paired blood-based clinical chemistries and metabolomics were obtained for 2,687 research consenting individuals that were formerly participants in the Arivale wellness program. Raw 16S amplicon sequence data were processed using QIIME2 (v2020.11.1). In brief, the QIIME2 workflow consisted of read demultiplexing using the command qiime tools import, and an associated manifest table for each study describing read metadata followed by read quality assessment using qiime demux summarize. Read quality assessment was used to determine trimming parameters for subsequent denoising using the QIIME2 implementation of DADA2 via the command, qiime dada2 denoise-single or qiime dada2 denoise-paired, for single and paired reads respectively. The first 10 bases were trimmed from all reads and reads were truncated to a length where median quality score was >20 (100-150 base pair for the data leveraged). Following denoising, data were reformatted into a table format using the command qiime metadata tabulate, and representative sequence taxonomy was inferred using a custom NCBI classifier with the command qiime feature-classifier classify-sklearn. The NCBI classifier was trained using 16S 515f-806r V4 regions extracted from all available bacterial NCBI genomes. To train the classifier 515f-806r, regions were extracted from NCBI sequences using the command qiime feature-classifier extract-reads, followed by the command qiime feature-classifier fit-classifier-naive-bayes using the extracted V4 sequences and a table of known taxonomies. For source code and tables of processed data refer to the Github repository listed below in Data and source code availability.

### Example 2: Model Construction and Growth Simulations

To construct community level metabolic models sample specific taxonomic abundance profiles inferred from 16S amplicon sequencing were summarized at the genus level and mapped to genus level metabolic models from the AGORA database (v1.03) using MICOM (v0.25.1). Genera with a relative abundance less than 0.1% were omitted from community models. An in silico media previously designed to represent an average western diet was applied which defined the bounds for metabolic imports by the model communities (refs 24, 26). Growth rates were then inferred using cooperative tradeoff flux balance analysis (ctFBA). In brief, this is a two-step optimization scheme, where the first step finds the largest possible biomass production rate for the full microbial community and the second step infers taxon-specific growth rates and fluxes, while maintaining community growth within a fraction of the theoretical maximum (i.e., the tradeoff parameter), thus balancing individual growth rates and the community-wide growth rate (ref 24). For all models herein a tradeoff parameter of 0.8 was used. This parameter value was chosen by identifying the largest tradeoff which allowed most (>90%) taxa to grow (growth rate>10-6). Import and export fluxes were estimated using parsimonious enzyme usage FBA (pFBA) and a defined medium constructed to represent an average European diet (ref 24). pFBA further constrained simulation results by requiring genera to utilize the lowest overall flux through their networks to achieve maximal growth (ref 23). For source code and tables of processed data refer to the Github repository listed below in Data and source code availability.

### Example 3: Probiotic Intervention

To model probiotic intervention a combination of strains previously shown to be effective at suppressing the growth of C. difficile in mice were used 9. Metabolic models for six of the eight stains in the VE303 cocktail described by Dsouza et al. were identified in the AGORA database and intervention was simulated by introducing them along with C. difficile to individual samples. a total probiotic fraction of 50% was used, which was evenly distributed among the six strains. This fraction was determined to be the most effective at suppressing the growth of C. difficile growth in silico for the samples tested (data not shown). Vancomycin treatment was simulated by reducing the abundance of C. difficile and all genera known to be impacted by vancomycin by 90% 45. Growth simulations were performed as described above. For source code and tables of processed data refer to the Github repository listed below in Data and source code availability.

### Example 4: Statistical Analysis

Statistical analyses were performed using functions from the python scipy (v1.7.1), seaborn (v0.11.2), sklearn-learn (v0.24.2), umap-learn (v0.5.1) and statsmodels (v0.13.1) packages. Linear associations were performed using the statsmodels ordinary least squares function OLS, and visualized using the seaborn function regplot. Least absolute shrinkage and selection operator (LASSO) was performed using the sklearn Lasso function and a training-test framework. Data were split into training and test sets (70% of samples were randomly assigned to the training set) and model performance was assessed across a range of regularization values spanning several orders of magnitude. LASSO training and test set R2 were used to select the model with the best test set R2 that did not overfit training data (training R2>>test R2). Analysis of variance (ANOVA) was performed using the statsmodels OLS and anova_lm functions. UMAP dimensionality reduction was performed using the umap function from the umap package and associated methods with default parameters (i.e., n_components=2, n_neighbors=15, metric=‘euclidean’, etc.). Biclustering was performed using the seaborn function clustermap and the Ward clustering algorithm. Hexagonal binning and associated histograms were generated using the seaborn function jointplot. Locally weighted scatterplot smoothing (LOWESS) curves were generated using the lowess function from statsmodels with default parameters. Additional statistical tests included the t-test and Wilcox rank sum test implemented in scipy as ttest_ind and wilcoxon respectively. For source code and tables of processed data refer to the Github repository listed below in Data and source code availability.

### Example 5: Data and Source Code Availability

Processed data tables and source code to reproduce the findings presented in this manuscript can be found at https://github.com/Gibbons-Lab/cdiff_invasion, which is hereby incorporated by reference in its entirety for all purposes. Raw 16S amplicon sequence data from David et al., Weingarden, A. et al., the American Gut (McDonald, D. et al.) can be downloaded using the sequence read archive (SRA) accession numbers PRJEB6518, PRJEB19996, and PRJEB11419 respectively. Metadata were obtained from manuscript supplementary information. Qualified researchers can access the full Arivale deidentified dataset, including all raw data, supporting the findings in this study for research purposes through signing a Data Use Agreement (DUA). Inquiries to access the data can be made at data-access@isbscience. org and will be responded to within 7 business days.

### Example 6: Development of an In Silico Invasion Assay to Simulate C. Difficile Colonization

To simulate the colonization of C. difficile an in silico invasion assay was developed that leverages microbiome relative abundance data, manually curated genome-scale metabolic models of gut bacteria from the AGORA database, and the MICOM modeling framework (refs 24, 26). Here, the focus was on leveraging available 16S amplicon sequencing data sets, which were by far more common than shotgun metagenomic data sets and provided a wider array of samples for validating the subject approach. Amplicon sequencing data is often limited to genus-level resolution in the taxonomic classifications of amplicon sequence variants (ASVs). Therefore, genus-level MCMMs (ref 30) were constructed for the invasion assays (see above methods). Specifically, strain-level metabolic models from AGORA were combined at the genus level, to account for potential coexistence of multiple strains and species from a given genus within an individual and to reduce potential bias from arbitrarily selecting individual strain models. Using this approach ˜80% of reads, on average, could be mapped to an NCBI genus-level taxonomic annotation across samples, and ˜75% of the total reads could be mapped to a genus-level metabolic model within the AGORA database (FIG. 6A). To simulate the invasion of C. difficile into these model communities, a pan-genus model of Clostridioides, representing four common C. difficile strains (including hypervirulent and non-epidemic strains), was introduced at a relative abundance of 10% (see below for justification of this percentage), while other community relative abundances were decreased proportionally to approximate a minor perturbation in community-wide biomass (FIG. 1A). Growth simulations were then performed using a medium representing an average European diet (i.e., a standard developed-world diet appropriate to the cohorts studied here), with fluxes of metabolites known to be absorbed in the small intestine decreased by 90%, as previously described (ref 24). Growth rates were estimated using ctFBA, as implemented in MICOM, which uses a regularization step and allows for a suboptimal community growth rate in order to achieve a more realistic growth rate distribution across the community (ref 24,25). Import and export fluxes were estimated using parsimonious enzyme usage FBA (pFBA) (ref 24).

Personalized MCMMs were constructed for each sample and the potential for C. difficile engraftment was quantified as the model-inferred growth rate. ctFBA has a single free parameter that needs to be chosen, the tradeoff between community-wide growth rates and individual, taxon-specific growth rates. Assuming that most genera detected at appreciable abundances in a gut microbiome are actively growing in vivo, a trade-off value was selected by choosing the minimal deviation from optimal community growth for which >90% of genera obtained non-zero growth rates on average (FIG. 6B). It was found that with a trade-off value of 0.8 (i.e., 80% of maximal community biomass production), the median fraction of genera with non-zero growth was >90%. Furthermore, at this tradeoff value, MCMM-inferred C. difficile growth rates accurately reflected trends in estimated C. difficile abundance across a time series with a known C. difficile colonization event (FIG. 1B) (refs 19, 31). Specifically, it was found that estimated C. difficile growth rates were at or below the limit of solver accuracy (<10-6, which effectively indicates a growth rate of zero) in samples collected prior to colonization and comparable to growth rates of other dominant genera in samples taken after the initial colonization event (FIG. 1B). Furthermore, patchy engraftment predictions were observed in a second individual that was known to be colonized by C. difficile at a low level (i.e., near the limit of detection) throughout the time series (FIG. 1C). The importance of propagule pressure (ref 32) (i.e., the relative abundance at which the invasive taxon is introduced into the models) was also assessed and found that below 10% relative abundance, agreement between growth rate estimates and measured abundances were poor (FIG. 6C). Thus, propagule pressure plays an important role in predicted engraftment success (ref 33). Based on these results, it was decided to use a fixed tradeoff value of 0.8 and a C. difficile invasion fraction of 10% for all subsequent analyses.

### Example 7: In Silico Invasion Assay Accurately Predicts C. difficile Colonization Potential in rCDI Subjects Pre- and Post-FMT

The in silico invasion model was applied to a dataset of rCDI subjects who received FMTs and were subsequently followed over time (ref 29). These data provided an additional validation of MCMM performance and a means to explore the metabolic features associated with community-scale colonization susceptibility or resistance across a larger population. Given that all individuals in the rCDI cohort had experienced multiple rCDI, it was expected the samples representative of subject pre-FMT microbiome to be susceptible to invasion. Additionally, Weingarden et al. showed that all the subject microbiomes returned to a compositional state more emblematic of healthy controls post-FMT (ref 29). Thus, it was expected post-FMT samples would be less susceptible to invasion but could show variation as a function of time. Subjects with rCDI, prior to FMT treatment, had significantly higher MCMM-predicted C. difficile growth rates compared to healthy individuals or to the same individuals after their FMT treatment (FIG. 2A; Welch's t-test p<0.01 for comparison of pre-FMT vs. post-FMT). Furthermore, predicted C. difficile growth rates were negatively associated with Shannon diversity, albeit weakly (FIG. 2B; ordinary least squares (OLS) R2=0.05, p=0.01), which is in line with prior empirical observations indicating that lower diversity communities are more susceptible to C. difficile colonization and the development of rCDI (refs 33-35).

The community-scale import flux profile prior to in silico invasion was predictive of C. difficile growth rate following invasion (FIG. 2C). High-dimensional community-scale import flux profiles were projected into a two-dimensional space using the Uniform Manifold Approximation and Projection (UMAP) technique (FIG. 2C) (ref 36). The UMAP projection provides a visual means of identifying patterns in the high dimensional import flux space. The closer points are to one another in this ordination the more similar their import flux profiles are. Thus, clusters of points in the UMAP can represent distinct metabolic environments across samples. The ordination plot indicated that C. difficile appears to grow well in more than one metabolic environment, when colonizing different individuals. Indeed, it was observed that the predicted metabolic environments occupied by C. difficile could vary within an individual over time (FIG. 2C). For most subjects, there was a transition from colonization-susceptibility pre-FMT to colonization-resistance post-FMT (FIG. 2A, C). Next, the different apparent niches that C. difficile was able to exploit when colonizing individuals in this CDI-FMT cohort was examined to better understand this phenotypic plasticity.

### Example 8: C. difficile is Predicted to Occupy Three Distinct Metabolic Niches Within the Human Gut Microbiome

To characterize C. difficile colonization-associated niches and identify the potential for multiple metabolic strategies associated with its growth, C. difficile import fluxes with high variance (log flux variance>=4.5) across the CDI-FMT cohort were examined. Biclustering of the high variance import flux data and an examination of how the apparent clusters associated with growth rates revealed that C. difficile makes use of multiple metabolic strategies (FIG. 3A). Three major clusters were observed across subject samples. These three clusters were designated as “high growth”, “moderate growth” and “no growth” (FIG. 3A). The high growth cluster included many of the pre-FMT samples and was characterized by consistently high import fluxes for all the metabolites identified as most strongly coupled to C. difficile growth across all models. The moderate growth cluster showed a sparser metabolite consumption profile. For example, ornithine and fructose were rapidly consumed in the high growth cluster, but showed almost no consumption in the moderate growth cluster (FIG. 3A). Very few metabolites were consumed by C. difficile above the zero-threshold of 10-6 in the no growth cluster (FIG. 3A).

The metabolic strategies employed by C. difficile within the MCMMs showed convergence with several observations from the literature. For example, it was found that metabolites known to promote growth of C. difficile in vivo (e.g., succinate, ornithine, and trehalose) were preferentially utilized when available and were associated with high pathobiont growth rates (refs 37-39). In addition, the consumption of the amino acids valine, glycine, glutamate, glutamine, and proline were associated with higher C. difficile growth rates in the MCMMs, indicating that C. difficile employs Strickland fermentation as one of its growth modes, which has been observed empirically (ref 40).

Following up on these findings, an examination of how cooperative and competitive interactions within MCMMs contributed to C. difficile colonization was carried out. To accomplish this, the import and export fluxes of metabolites associated with C. difficile colonization (e.g., amino acids, ornithine, succinate, etc.) were examined. Genera that produced metabolites consumed by C. difficile likely promote its growth, while those consuming C. difficile growth-associated metabolites may be in direct competition. For ornithine and succinate, it was found that cooperative and competitive interactions are context-dependent, varying across samples. The genus Phocaeicola, for instance, produces ornithine in some samples, which is in turn consumed by C. difficile, while in other contexts it consumes ornithine, competing with C. difficile (FIG. 3B). Meanwhile, Roseburia, and Faecalibacterium compete with C. difficile for ornithine, but these genera also produce succinate and cysteine in some contexts, which C. difficile consumes (FIG. 3B). Thus, community context is an important factor in determining the metabolic strategies used by C. difficile and can lead to competitive or cooperative interactions, which may hinder or promote colonization.

Finally, it was assessed if compositional variation in the microbiome could explain observed differences in predicted C. difficile growth rate. Compositional variation was found to be a modest predictor of estimated C. difficile growth rate (out of sample R2=0.37 using best least absolute shrinkage and selection operator (LASSO) regression fit to the CDI-FMT cohort, see Methods). Meanwhile, the import flux derived clusters (e.g., “high growth”, “medium growth”, and “no growth” groups) explained the vast majority of the variance in predicted C. difficile growth rates (analysis of variance (ANOVA) R2=0.94 using the CDI-FMT cohort), suggesting that composition alone may not be sufficient for accurate engraftment predictions.

### Example 9: Associations with C. difficile Growth Provides Insights into the Community Context

In order to assess the consistency of the C. difficile growth clusters, four independent data sets were leveraged, including the time series and CDI-FMT studies presented above (FIGS. 1-3), along with two large cross-sectional cohorts (i.e., the American Gut and Arivale cohorts), covering a total of 14862 individuals(refs 27, 31, 41, 42). Growth and flux predictions generated across all four data sets were evaluated and found that C. difficile fell into the same three clusters as identified in the CDI-FMT data set, representing no growth, moderate growth, and high growth (FIG. 4A).

To further contextualize the metabolic niche of C. difficile, model outputs for all four data sets were integrated. Specifically, import fluxes across all genera were examined. Most genera formed unique clusters in the UMAP projection, suggesting that each genus had a single metabolic niche that was consistent across datasets (FIG. 4B). Within this community context, it was found that C. difficile still fell into three distinct clusters (FIG. 4B). Three genera that showed some of the strongest competitive and cooperative interactions with C. difficile, Blautia, Faecalibacterium, and Eubacterium, clustered near to one another in import flux space, indicating that these taxa had a similar metabolic niche (FIG. 4B). However, these same taxa clustered apart from C. difficile in their overall import flux profiles, with the exception of a few scattered samples (FIG. 4B).

Next, gut community diversity and predicted C. difficile growth rates across the four data sets were explored. Specifically, Shannon diversity was examined, which integrates species richness and evenness and is commonly used to quantify gut microbiome alpha-diversity. Lower Shannon diversity is commonly associated with disease states, like diarrhea, while higher diversity has generally been associated with diverse plant-based diets and overall better health (ref 34). However, constipated individuals generally have higher gut microbiome alpha-diversity as well, suggesting that there may be an optimal range of alpha-diversity across healthy individuals (ref 43). An initial analysis using the CDI-FMT cohort suggested a negative linear relationship between predicted C. difficile growth rate and Shannon (FIG. 2B). However, the integrated data sets, which spanned a wider range of diversity, showed a U-shaped relationship between Shannon diversity and predicted C. difficile growth rate (FIG. 4C). Intermediate levels of Shannon diversity were associated with the lowest predicted growth rates, on average, with higher average growth at the upper and lower tails of the distribution (FIG. 4C). The relationship between Shannon diversity and predicted growth rate suggests extremes in either direction on the diversity scale are, on average, more permissive to C. difficile engraftment.

### Example 10: Blood Metabolites and Clinical Labs Associated with MCMM-Predicted C. difficile Colonization Susceptibility

Next, it was sought to identify potential blood-based markers that were significantly associated with MCMM-predicted C. difficile growth rate. Previous work has shown that circulating blood metabolites can be leveraged to predict gut microbiome alpha-diversity (ref 41). Several blood metabolites and clinical chemistries were identified as being significantly associated with C. difficile growth rate, after adjusting for common covariates (i.e., sex, age, and BMI) and correcting for multiple tests (FDR q<0.05). These included two secondary bile acids, an unannotated metabolite previously associated with the abundance of the family Eggerthellacea, and several red blood cell-associated clinical chemistries (FIG. 6D) (ref 44). Unfortunately, while significant, these blood-based markers, along with sex, age, and BMI, collectively accounted for only ˜5% of the variance in MCMM-predicted growth rates. Thus, it appears MCMM-based estimates of C. difficile engraftment cannot be readily replaced with commonly measured clinical chemistries or blood metabolites.

### Example 11: MCMMs Predict Engraftment Heterogeneity of Probiotic Cocktail Designed to Treat rCDI

As a proof-of-concept for the modeling framework, a probiotic intervention was simulated using a previously validated probiotic cocktail designed to treat rCDI (ref 9). The probiotic, referred to as VE303, was composed of 8 commensal Clostridia strains and shown to be effective at treating CDI in mice (refs 9, 10). This probiotic was also shown to be safe and well-tolerated and effective in reducing rCDI incidence in humans (refs 9, 10). Furthermore, the authors demonstrated that, for effective engraftment, administration of the probiotic needed to occur following antibiotic treatment (ref 9). With these facts in mind, a simulated intervention was configured that mimicked the treatment found to be most effective by Dsouza et al. Metabolic models for 6 of 8 strains in VE303 in the AGORA database (ref 26) were identified. The CDI-FMT dataset was leveraged to test this six-member probiotic cocktail, paired with in silico invasion by C. difficile. The probiotic cocktail was introduced to subject samples, alongside 10% C. difficile, at a total relative abundance of 50%, which was evenly distributed among the six strains. Vancomycin treatment was simulated by reducing the abundance of C. difficile and all commensal genera known to be impacted by vancomycin (ref 45) by 90%. As demonstrated in FIG. 5A, it was found that a combined probiotic and antibiotic intervention most effectively suppressed the growth of C. difficile in both the moderate and high C. difficile growth rate clusters.

To better understand the mechanism of action of the probiotic cocktail, growth characteristics and the niche proximity of the probiotic strains were assessed in relation to C. difficile. As illustrated in FIG. 5B, it was found that suppression of C. difficile growth occurred when the average growth of the probiotic strains was high (>10-4) and when the average niche distance between the probiotic strains and C. difficile was low (<25, FIG. 5C). Further, relative to other genera, several of the probiotics strains occupied niches closer to C. difficile (FIG. 7). A comparison was also performed across the import fluxes of the probiotic strains and C. difficile for the metabolites identified as important for C. difficile growth (FIG. 3). This analysis showed that, in addition to occupying niches similar to C. difficile, several of the probiotic strains directly competed for metabolites important for C. difficile growth, such as succinate, ornithine, and trehalose (FIG. 5D). Cumulatively these results suggest that metabolic competition is the mechanism by which the probiotic cocktail suppressed C. difficile growth, as was suggested in the original study 9. Results indicated that certain probiotic strains were more or less likely to engraft in an individual (FIG. 5D), and that this engraftment/growth was associated with C. difficile suppression (FIG. 5B), which indicates that MCMMs can be leveraged to identify responders and non-responders prior to these kinds of probiotic interventions.

### Example 12: Discussion

In this study, a framework for predicting C. difficile engraftment risk in the human gut microbiome using MCMMs was provided. While this example focuses on C. difficile, due to its clinical importance, this approach could be extended to other opportunistic bacterial pathogens, probiotic organisms, or even entire communities, in the case of FMTs. The results from the Example demonstrate how the disclosed approach predicts expected longitudinal and cross-sectional variation in C. difficile colonization potential, and insights are provided regarding the metabolic strategies leveraged by C. difficile in different ecological contexts. The analysis not only recapitulates known metabolic associations with C. difficile growth (e.g., consumption of trehalose, ornithine, and succinate; FIG. 3), it also suggests additional associations (e.g., importance of reduced sulfur compounds like cysteine, Stickland fermentation reactants, and utilization of other sugars, like fructose; FIG. 3). Additionally, results disclosed herein demonstrate that competition and cooperation with community members can prevent or promote colonization of C. difficile, and that many of these associations are highly context-dependent (FIG. 3).

Supporting the idea that simple metrics of community structure and composition alone are not effective predictors of colonization susceptibility, results from this Example indicated that community compositional variation was a modest predictor of estimated C. difficile growth rate and the relationship between alpha diversity and estimated C. difficile growth rate was nonlinear (FIG. 4C). Not only did low diversity communities tend to be more invasible, as might be expected due to non-saturation of the metabolic niche space, but high diversity communities were also more prone to C. difficile engraftment. In high diversity communities, successful invasion may be due the construction of new niches or changes in the interaction landscape that are in line with the diversity-begets-diversity hypothesis 46. Thus, an intermediate range of alpha-diversity appears to be optimal for mitigating C. difficile colonization potential (FIG. 4C). Overall, these complex mappings between community composition and pathobiont engraftment risk underscore the necessity of systems-scale tools, like MCMMs, that are capable of synthesizing this complexity.

Several genera were identified that engage in cooperative and competitive interactions with C. difficile across MCMMs. Blautia, Faecalibacterium, and Eubacterium were all shown to benefit C. difficile through production of key metabolites that it consumes, like succinate, but were also capable of competition for metabolic resources (FIG. 3). Meanwhile Rumminococcus, Bacteroides and Phocaeicola were more often competing for the same metabolites that C. difficile consumed (FIG. 3). Contextualizing these results through analysis of individual taxon import fluxes across studies, Blautia, Faecalibacterium, and Eubacterium share similar niches with one another. In most cases, these niches did not overlap with C. difficile, but in a subset of individuals all three occupied niches states in close proximity to C. difficile (FIG. 4B). Thus, while competition for some key metabolites was observed, on a global scale the majority of the metabolic niche space used by C. difficile tends not to overlap with its apparent competitors (FIG. 4B). These results highlight how flexible commensal gut bacteria are in adapting their import fluxes to the communities in which they reside, which in turn suggests why so many taxa are able to coexist.

In addition to developing a simulation framework to predict engraftment blood-based clinical chemistries and blood metabolites that were associated with MCMM-inferred C. difficile growth rate were estimated. Three blood metabolites that were independently associated with predicted C. difficile growth rates were identified. These included two secondary bile acids and an unannotated metabolite. One of the secondary bile acids, isoursodeoxycholate, has previously been positively associated with the abundance of Bacteroides (ref 41), and was negatively associated with predicted C. difficile growth rate. This result is in line with the apparent competition between Bacteroides and C. difficile in our MCMMs. Several clinical labs negatively associated with predicted growth rate were also identified (FIG. 6D). However, together with age, sex, and BMI, these features only accounted for ˜5% of the variance in predicted growth rates. Thus, while these features may be signatures for colonization susceptibility in the blood, their clinical relevance is limited at this time.

It was also determined that a probiotic intervention (VE303), which recently showed positive efficacy results in a double-blinded, placebo-controlled clinical trial for the treatment of rCDI (ref 10), suppresses the growth of C. difficile in silico in most people (FIG. 5), which supports prior work showing C. difficile growth suppression by this cocktail in mice (ref 9). We also showed that the mechanism of action for this particular probiotic is likely competition for metabolites essential for the growth of C. difficile, as many of the probiotic strains occupy niches close to C. difficile and directly compete for metabolites, such as succinate and ornithine, in samples where growth suppression was observed (FIG. 5). Furthermore, analysis of niche distances between C. difficile and other genera across donors suggest selecting strains from Blautia and Dorea (e.g., B. producta and D. longicatana, from VE303), in addition to Anaerostipes, Roseburia, and Faecalibacterium, could be leveraged to design individual-specific probiotic cocktails capable of suppressing C. difficile and rescuing the VE303 non-responders (FIGS. 7 and 5B). These results illustrate how MCMMs are powerful tools for assessing the individual-specific efficacy of clinically-relevant probiotics, in addition to understanding personalized pathobiont colonization susceptibility.

In view of the many possible embodiments to which the principles of the disclosure may be applied, it should be recognized that the illustrated embodiments are only examples and should not be taken as limiting the scope of the invention.

Some embodiments of the present disclosure include a system including one or more data processors. In some embodiments, the system includes a non-transitory computer readable storage medium containing instructions which, when executed on the one or more data processors, cause the one or more data processors to perform part or all of one or more methods and/or part or all of one or more processes disclosed herein. Some embodiments of the present disclosure include a computer-program product tangibly embodied in a non-transitory machine-readable storage medium, including instructions configured to cause one or more data processors to perform part or all of one or more methods and/or part or all of one or more processes disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention as claimed has been specifically disclosed by embodiments and optional features, modification and variation of the concepts herein disclosed may be resorted to by those skilled in the art, and that such modifications and variations are considered to be within the scope of this invention as defined by the appended claims.

The present description provides preferred exemplary embodiments only, and is not intended to limit the scope, applicability or configuration of the disclosure. Rather, the present description of the preferred exemplary embodiments will provide those skilled in the art with an enabling description for implementing various embodiments. It is understood that various changes may be made in the function and arrangement of elements without departing from the spirit and scope as set forth in the appended claims.

Specific details are given in the present description to provide a thorough understanding of the embodiments. However, it will be understood that the embodiments may be practiced without these specific details. For example, circuits, systems, networks, processes, and other components may be shown as components in block diagram form in order not to obscure the embodiments in unnecessary detail. In other instances, well-known circuits, processes, algorithms, structures, and techniques may be shown without unnecessary detail in order to avoid obscuring the embodiments.

What is claimed is:

1. A computer-implemented method for determining bacterial or pathobiont engraftment, comprising:
(a) accessing taxon abundance data of a gut microbiome of a subject;
(b) accessing a model configured to predict—using a flux balance analysis—dynamics of individual taxa of the gut microbiome of the subject and of engraftment bacteria or engraftment pathobiont, the model constrained by:
(i) growth medium data representing extracellular substrate availability; and
(ii) relative taxon abundance comprising the taxon abundance data of the gut microbiome of the subject in combination with taxon abundance of the engraftment bacteria or engraftment pathobiont set at a propagule pressure approximating an exposure or infection event;
(c) generating a prediction of an engraftment potential for the engraftment bacteria or engraftment pathobiont by processing the taxon abundance data of the gut microbiome of the subject using the model; and
(d) outputting the prediction of the engraftment potential of the engraftment or engraftment pathobiont bacteria for the subject.

2. The method of claim 1, wherein the model is augmented with an intervention comprising one or more antimicrobials, prebiotics, probiotics, fecal microbiota transplants, dietary interventions, or a combination thereof.

3. The method of claim 2, wherein the probiotic and the fecal microbiota transplant interventions comprise treatment bacteria, and wherein the model is a microbial community-scale metabolic network model comprising a plurality of metabolic models for the individual taxa augmented with one or more metabolic models for the treatment bacteria having a taxon abundance approximating a gut exposure of interest.

4. The method of claim 2, wherein an antimicrobial intervention comprises one or more antibiotics, and wherein the taxon abundance of one or more susceptible taxa of the model are modified so as to approximate the antimicrobial activity of the one or more antibiotics.

5. The method of claim 4, wherein the antibiotic is selected from metronidazole, vancomycin, and fidaxomicin, and the antimicrobial activity is about half maximal effective concentration or greater.

6. The method of claim 2, wherein the prebiotics and the dietary interventions augment the growth medium data in an amount approximating a relative dosage of interest.

7. The method of claim 2, wherein the prebiotic intervention comprises, or is selected from, soluble fiber such as inulin, pectin and psyllium, and insoluble fiber such as wheat bran, cellulose, lignin, and resistant starch, and the dietary intervention comprises, or is selected from, food intake, minerals, and vitamins.

8. The method of claim 1, wherein the growth medium is constrained by diet such as food type and food quantity, host metabolism such as by absorption of growth medium material in the small intestines, and one or more additional substrates selected from host molecules such as mucins and bile acids, vitamins, minerals, and prebiotics such as pectin and inulin.

9. The method of claim 2, further comprising:
generating an intervention efficacy score by comparing the predicted engraftment potential of the engraftment bacteria or engraftment pathobiont with and without the intervention.

10. The method of claim 9, wherein the intervention efficacy score comprises a ratio of the predicted engraftment potential of the engraftment bacteria or engraftment pathobiont with and without the intervention.

11. The method of claim 1, wherein the predicted engraftment potential comprises:
growth rate; or
taxon abundance relative to a combination of the gut microbiome of the subject and the gut microbiome of the growth medium.

12. The method of claim 1, wherein the propagule pressure approximating an exposure or infection event is about 10% of the relative taxon abundance data.

13. The method of claim 1, further comprising:
(e) displaying the predicted engraftment potential of the subject relative to an engraftment potential of a reference population.

14. The method of claim 1, wherein the flux balance analysis is cooperative tradeoff flux balance analysis.

15. The method of claim 1, wherein an objective function for the flux balance analysis is configured to reward: community-wide growth corresponding to a full microbial community and taxon-specific growth specific to a given taxon.

16. The method of claim 1, wherein an objective function for the flux balance analysis is configured to reward: community-wide growth corresponding to a full microbial community and taxon-specific growth specific to a given taxon and production of short-chain fatty acids.

17. The method of claim 1, wherein the engraftment bacteria or engraftment pathobiont is one of: pathobiont bacteria, probiotic bacteria, fecal microbiota transplant (FMT) bacteria, or a combination thereof.

18. The method of claim 1, wherein the engraftment bacteria or engraftment pathobiont comprise Clostridioides difficile or a mixture of strains thereof.

19. The method of claim 18, wherein the Clostridioides difficile or a mixture of strains thereof comprise, or are selected from, a pan-genus model of Clostridioides representing common hypervirulent and non-epidemic strains, such as Clostridium difficile CD196, NAP07, NAP08, and R20291.

20. The method of claim 17, wherein the probiotic bacteria or engraftment pathobiont comprise human gut commensal bacteria or a mixture of strains thereof.

21. The method of claim 20, wherein the human gut commensal bacteria or a mixture of strains thereof are selected from Enterocloster bolteae, Anaerotruncus colihominis, Sellimonas intestinalis, Clostridium_Q symbiosum, Blautia sp001304935, Dorea_A longicatena, Clostridium_AQ innocuum, Flavonifractor plautii, Anaerobutyricum soehngenii, Akkermansia muciniphila, Anerobutyricum hallii, Clostridium beijernckii, Clostridium butyricum, Bifidobacterium infantis, and Generally Recognized as Safe (GRAS) bacterial strains.

22. The method of claim 17, wherein the fecal microbiota transplant (FMT) bacteria comprise, or are selected from, OpenBiome FMTs.

23. The method of claim 1, wherein step (d) further comprises outputting metabolite uptake and metabolite secretion of the engraftment bacteria or engraftment pathobiont relative to the gut microbiome of the subject and the growth medium.

24. The method of claim 1, wherein the model is a microbial community-scale metabolic network model (MCMM) generated by mapping the taxon abundance data of the subject to a plurality of metabolic models of the MCMM corresponding to the individual taxa of the subject.

25. A system comprising:
one or more data processors; and
a non-transitory computer readable storage medium containing instructions which, when executed on the one or more data processors, cause the one or more data processors to perform a set of actions comprising:
(a) accessing taxon abundance data of a gut microbiome of a subject;
(b) accessing a model configured to predict—using a flux balance analysis—dynamics of individual taxa of the gut microbiome of the subject and of engraftment bacteria or engraftment pathobiont, the model constrained by:
(i) growth medium data representing extracellular substrate availability; and
(ii) relative taxon abundance comprising the taxon abundance data of the gut microbiome of the subject in combination with taxon abundance of the engraftment bacteria or engraftment pathobiont set at a propagule pressure approximating an exposure or infection event;
(c) generating a prediction of an engraftment potential for the engraftment bacteria or engraftment pathobiont by processing the taxon abundance data of the gut microbiome of the subject using the model; and
(d) outputting the prediction of the engraftment potential of the engraftment or engraftment pathobiont bacteria for the subject.

26. A computer-program product tangibly embodied in a non-transitory machine-readable storage medium, including instructions configured to cause one or more data processors to perform a set of actions comprising:
(a) accessing taxon abundance data of a gut microbiome of a subject;
(b) accessing a model configured to predict—using a flux balance analysis—dynamics of individual taxa of the gut microbiome of the subject and of engraftment bacteria or engraftment pathobiont, the model constrained by:
(i) growth medium data representing extracellular substrate availability; and
(ii) relative taxon abundance comprising the taxon abundance data of the gut microbiome of the subject in combination with taxon abundance of the engraftment bacteria or engraftment pathobiont set at a propagule pressure approximating an exposure or infection event;
(c) generating a prediction of an engraftment potential for the engraftment bacteria or engraftment pathobiont by processing the taxon abundance data of the gut microbiome of the subject using the model; and
(d) outputting the prediction of the engraftment potential of the engraftment or engraftment pathobiont bacteria for the subject.

27. A computer-implemented method for determining bacterial or pathobiont engraftment, comprising:
(a) accessing engraftment potential data for:
(i) a gut microbiome of a subject simulated on a model configured to predict—using a flux balance analysis—dynamics of individual taxa of the gut microbiome of the subject and of engraftment bacteria or engraftment pathobiont, the model constrained by
(1) relative taxon abundance comprising taxon abundance data of the gut microbiome of the subject in combination with taxon abundance of the engraftment bacteria or engraftment pathobiont set at a propagule pressure approximating an exposure or infection event,
(2) growth medium data representing extracellular substrate availability from one or more different background diets, and
(3) no intervention, or one or more interventions, the interventions comprising one or more antimicrobials, prebiotics, probiotics, fecal microbiota transplants, dietary interventions, or a combination thereof; and
(ii) a plurality of gut microbiomes of a reference population comprising generally healthy individuals each individually simulated for engraftment potential on essentially the same one or more background diets as the subject, and optionally, essentially the same interventions as the subject;
(b) generating, for each of the one or more different background diets, a distribution based on engraftment potential of the subject and the reference population associated with the background diet and embedding the engraftment potential data of the subject associated with the background diet into a context of the distribution;
(c) generating, for each of the one or more different background diets, a comparative metric using the distribution for the background diet and the engraftment potential data of the subject simulated on the background diet; and
(d) identifying, based on the comparative metrics, a particular intervention to recommend for the subject, where the particular intervention includes a particular background diet, one or more particular interventions, or a combination thereof.

28. The method of claim 27, wherein the comparative metric is for a plurality of different background diets with and without the one or more interventions.

29. The method of claim 27, further generating a gut health report embedding the engraftment potential of the subject into a context of the distribution of the engraftment potential of the reference population for a given background diet, the gut health report identifying the particular intervention.

30. The method of claim 27, wherein the identifying the particular intervention comprises ranking the interventions based on background diet.

31. The method of claim 27, wherein the background diets comprise, or are selected, from a high-fiber diet such as a vegan high-fiber diet rich in resistant starch or a standard Mediterranean diet, a low fiber diet such as a standard European diet or a standard American diet, and a personalized diet.

32. A system comprising:
one or more data processors; and
a non-transitory computer readable storage medium containing instructions which, when executed on the one or more data processors, cause the one or more data processors to perform a set of actions comprising steps (a)-(d) of claim 27.

33. A computer-program product tangibly embodied in a non-transitory machine-readable storage medium, including instructions configured to cause one or more data processors to perform a set of actions comprising steps (a)-(d) of claim 27.
